(19) United States
(12) Patent Application Publication
(10) Pub. No.: US 2017/0137480 A1
(43) Pub. Date: May 18, 2017

(54) RPGR-OFR15 VARIANT GENE IN THE TREATMENT OF RETINITIS PIGMENTOSA

(71) Applicant: UCL BUSINESS PLC, Greatere London (GB)

(72) Inventors: Robin ALI, Greater London (GB); Alexander SMITH, Greater London (GB)

(21) Appl. No.: 15/323,770

(22) PCT Filed: Jul. 6, 2015
(86) PCT No.: PCT/GB2015/051956

§ 371 Date: Jan. 4, 2017

(30) Foreign Application Priority Data
Jul. 4, 2014 (GB) 1412011.7

(51) Int. Cl. C07K 14/47; C12N 15/86

(52) U.S. Cl. CPC C07K 14/4702; C12N 15/86; C12N 2750/14143

(57) ABSTRACT

The present invention relates to the treatment of ocular disease, specifically retinitis pigmentosa, by gene therapy using a modified version of the RPGR-ORF15 gene.

### FIELD OF THE INVENTION

The present invention relates to the treatment of ocular disease, specifically retinitis pigmentosa, by gene therapy.

### BACKGROUND TO THE INVENTION

X-linked retinitis pigmentosa type 3 (XRP3), caused by mutations in the RPGR (retinitis pigmentosa GTPase regulator) gene, is the most common form of inherited retinal dystrophy with a prevalence of around 1 in every 15,000 boys. Patients usually experience loss of peripheral vision and reduced central vision by the age of 20, accompanied by significant changes to pigmentation in the retina that are characteristic of retinitis pigmentosa. The relative severity of the disorder and the recessive pattern of inheritance make XRP3 a suitable target for development of a treatment using gene therapy vectors.

The RPGR gene is expressed in many tissues, but an isoform exists that is preferentially expressed in the retina (RPGR-ORF15). This isoform includes a region of intron 15 containing a 1500 nucleotide tract of predominantly purines. The RPGR-ORF15 protein is localised in the connecting cilium of the rod and cone photoreceptor cells, where it is involved in the transport of components of the phototransduction cascade to the outer segments.

The development of XRP3 gene therapy has been hindered by the long stretch of poly-purine DNA that makes RPGR-ORF15 cDNA unstable, rendering it undesirable for clinical use, due to the resultant uncertainty about the clinical product.

In 2005, the group of Tiansen Li published a paper that showed that a murine RPGR-ORF15 cDNA with a 654 bp in-frame deletion in the purine tract can successfully restore RPGR activity and slow degeneration in RPGR−/− mice. This was done by crossing the knock-out mice with a transgenic mouse line expressing the above cDNA (Hong et al (2005) IOVS 46: 435-441).

### SUMMARY OF THE INVENTION

We have created a modified version of the RPGR-ORF15 gene (RPGR-ORF15*) that has 456 base pairs (152 amino acids) of the highly repetitive sequence removed to improve its stability. This construct was used to create AAV2/5 and AAV2/8 vectors that were used to treat RPGR−/− mice via a subretinal injection of therapeutic vector into one eye and an injection of control vector into the contralateral eye. The produced protein localises correctly to the connecting cilia of the photoreceptor cells. Western blot analysis shows that the protein is of the expected size, indicating that the vector is indeed stable. Electroretinography (ERG) analysis of retinal responses to light indicated that the rod photoreceptor cells in the eyes that were treated with this new therapeutic gene therapy vector were more sensitive to light than photoreceptors from eyes treated with a control vector, indicating that the construct was functional in vivo.

We have therefore shown that stabilization of the gene via our deletion is effective and that the deleted sequence is not necessary for rescue of function. Using this modified RPGR gene, we have therefore created an AAV gene therapy vector that is suitable for production to clinical grade standards, offering a way to treat this highly debilitating disease in humans by gene therapy.

Accordingly, the invention provides

A polynucleotide comprising:
(a) the nucleotide sequence shown in SEQ ID NO: 5;
(b) a nucleotide sequence that encodes the polypeptide sequence of SEQ ID NO: 6;
(c) a nucleotide sequence comprising the sequence of SEQ ID NO: 1 but with a deletion corresponding to (i) the sequence of SEQ ID NO:3, (ii) the sequence of SEQ ID NO: 3 and up to 75 additional nucleotides flanking SEQ ID NO:3 on one or both sides of SEQ ID NO:3 in the sequence of SEQ ID NO: 1, or (iii) 390 or more contiguous nucleotides from within SEQ ID NO:3; or
(d) a nucleotide sequence according to (a), (b) or (c) but truncated at one or both ends by up to 150 nucleotides per end.

said polynucleotide having the ability to rescue loss of RPGR (retinitis pigmentosa GTPase regulator) function.

The invention also provides polypeptides encoded by polynucleotides of the invention.

The invention also provides vectors in which a polynucleotide of the invention is operably linked to a promoter, as well as pharmaceutical compositions comprising such vectors and the use of such vectors in treatment of retinitis pigmentosa.

### BRIEF DESCRIPTION OF THE FIGURES

FIG. 1: (A) Schematic representation of the wildtype (top) and the modified (bottom) RPGR cDNA. The 3459 nt wildtype RPGR cDNA contains a repetitive sequence (shown in darkest shading) that affects its stability. Removal of 456 base pairs (nt 2485-nt 2940) from this repetitive sequence stabilises the construct. The modified cDNA (bottom) is sufficiently stable to be delivered using gene therapy vectors. (B) Human RPGR-ORF15 full length (SEQ ID NO: 1/2) sequence (3.5 kbp), showing (bold, underlined) the fragment of cDNA that is deleted (SEQ ID NO: 3/4) from the stabilized construct of the invention to generate the modified RPGR-ORF15* sequence of the invention (SEQ ID NOS: 5/6).

FIG. 2: Western blot analysis of retinas injected with the modified RPGR-ORF15 vector show RPGR protein of the expected size (˜170 kD), which is absent from the control injected retina, showing that the new construct is stable.

FIG. 3: Staining of RPGR protein in retinas of RPGR-deficient mice treated with the AAV2/8.RPGR-ORF15* vector shows that the protein correctly localises to the connecting cilia of the photoreceptor cells. No staining is present in untreated eyes.

FIG. 4: ERG analysis of eyes injected with therapeutic vector AAV2/8.RPGR-ORF15*(solid lines) and control vector AAV2/8.GFP(dotted lines). Lower scotopic a-wave values (left) in the eyes treated with RPGR-ORF15* indicate a greater rod activity in response to light. Higher scotopic b-wave values (right) indicate that there is a greater activity of the inner retinal neurons in response to rod function.

FIG. 5: Schematics of therapeutic and control vectors used herein.

### BRIEF DESCRIPTION OF THE SEQUENCES

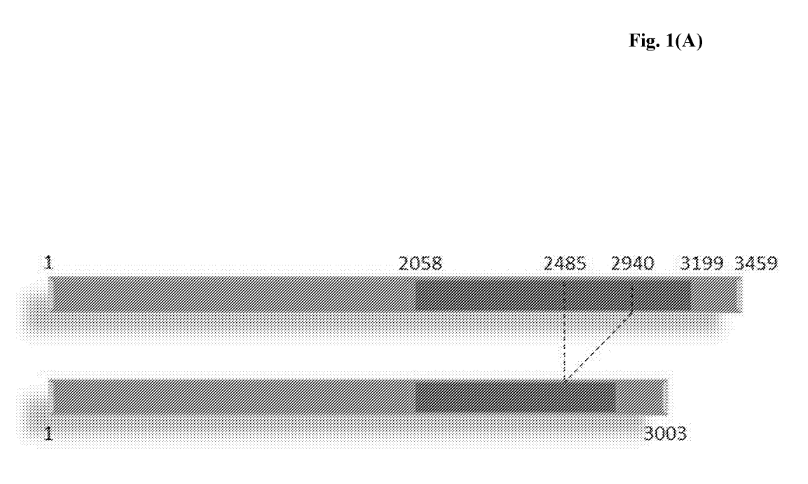

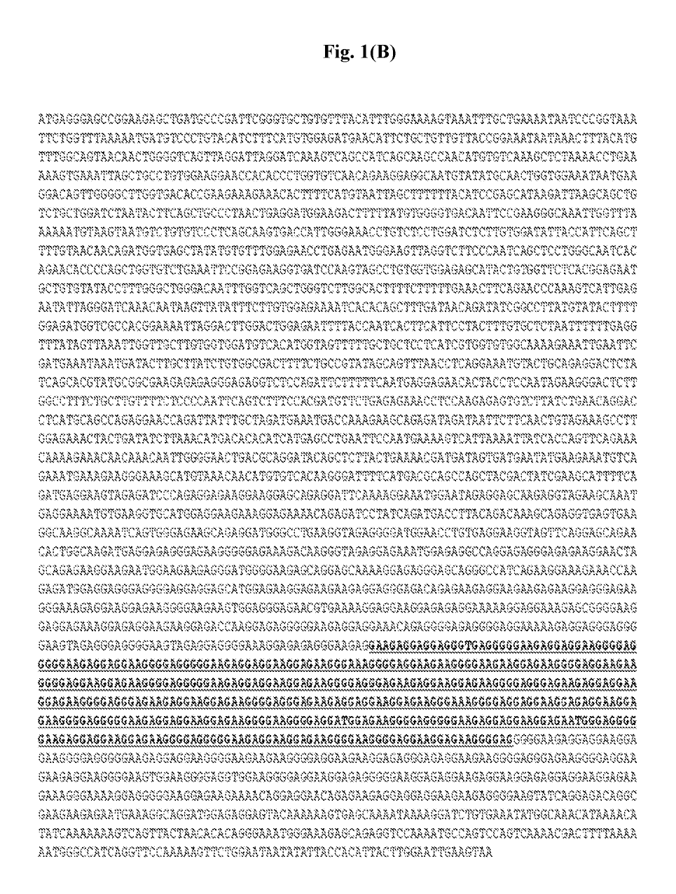

SEQ ID NOs: 1/2: Full-length human DNA (SEQ ID NO: 1) and amino acid sequence (SEQ ID NO: 2) of RPGR-ORF15 gene (See also FIG. 1B).

SEQ ID NOs: 3/4: DNA (SEQ ID NO: 3) and amino acid (SEQ ID NO: 4) sequence of deletion from within RPGR-ORF15 that leads to RPGR-ORF15* sequence of present invention.

SEQ ID NO: 5/6: DNA (SEQ ID NO: 5) and amino acid sequence (SEQ ID NO: 6) RPGR-ORF15* sequence of present invention.

### DETAILED DESCRIPTION OF THE INVENTION

Polynucleotide Sequences of the Invention

The invention provides the RPGR-ORF15* sequence of SEQ ID NO: 5 and variants and derivatives of this sequence that retain its beneficial properties (see below).

SEQ ID NO: 5 arises from a deletion of the sequence of SEQ ID NO: 3 (456 nucleotides) from within the sequence of SEQ ID NO: 1. The invention also encompasses sequences similar to SEQ ID NO: 5 with somewhat different deletions. For example, up to 5, 10, 20, 50, 75, or 100 extra flanking nucleotides could be deleted at one or both ends of SEQ ID NO: 3, ie at the 5′ end or the 3′ end or both. Or the deletion can be smaller rather than larger than SEQ ID NO: 3, eg the deletion may correspond to any 390, 400, 420 or 450 or more nucleotides of SEQ ID NO: 3.

The sequence of SEQ ID NO: 5 may be truncated at one or both ends, either the 5′ end or the 3′ end of SEQ ID NO: 5 or both, by up to 10, 20, 50, 100, 150, 200, 300, 400 or 500 nucleotides.

The invention also encompasses sequences which, by virtue of the degeneracy of the genetic code, encode the same polypeptide as any of the above.

Variations from the sequence of SEQ ID NO: 5 may be of any type, eg deletions, substitutions or insertions. They may or may not affect the sequence of the encoded polypeptide. If they introduce substitutions into the the coding sequence, these may be conservative or non-conservative substitutions. However, sequences of the invention will typically remain “in frame” and avoid stop codons, such that they encode a sequence comparable in length with SEQ ID NO: 6.

Polypeptide Sequences of the Invention

Polypeptide sequences of the invention are encoded by polynucleotide sequences of the invention as defined above. Polypeptide sequences of the invention are typically expressed in vivo using gene therapy vectors as described herein but can also be produced and recovered in vitro by standard recombinant expression techniques.

Properties of Sequences of the Invention

Polynucleotide sequences of the invention have the ability to rescue loss of RPGR function, which may occur for example by mutations in the RPGR gene. “Rescue” generally means any amelioration or slowing of progression of a XRP3 disease phenotype, for example restoring presence of RPGR-ORF15 protein in the connecting cilium, restoring or improving transport through the connecting cilium, improving ERG activity or slowing loss of ERG activity, improving retinal sensitivity or slowing/halting progressive loss of retinal sensitivity, slowing or halting loss of photoreceptor cells, improving vision or slowing/halting vision loss.

The properties of sequences of the invention can also be tested using techniques based on those in the Examples. In particular, a sequence of the invention can be assembled into a vector of the invention and delivered to the retina of an RPGR-deficient test animal, such as a mouse, and the effects observed and compared to a control. Preferably, the control will be the other eye of the same mouse, which is either untreated or treated with a control vector such as one containing a reporter gene as opposed to a sequence of the invention. Electroretinography analysis of retinal responses to light can then be used to confirm that photoreceptor cells in the eyes that are treated with are more sensitive to light than photoreceptors from eyes that are untreated or treated with a control vector. The sensitivity of the treated eye to light may for example be at least 1.1, 1.2, 1.5, 2, 5, 10, 20, 50 or 100-fold greater than that of the untreated or control-treated eye.

Vectors

The sequences of the invention can be placed in any suitable vector and will typically be operably linked to a promoter. The vector of the invention may for example be a plasmid vector but viral vectors such as lentivirus, adenovirus and adeno-associated virus (AAV) vectors are preferred. AAV vectors are particularly preferred. Any suitable AAV may be used but AAV2/5 (AAV2 genome pseudotyped with an AAVS capsid) or AAV2/8 (AAV2 genome pseudotyped with AAV8 capsid) are two preferred examples.

Promoters and Other Vector Components

In vectors of the invention, any suitable promoter may be used. Suitable promoters may be constitutive or tissue-specific. Constitutive promoters include the CMV, SV40 and ubiquitin promoters. Photoreceptor cell-specific promoters are preferred, for example the human rhodopsin kinase (GRK1) promoter.

Vectors may also contain other standard components, particularly polyadenylation (polyA) signals. The SV40 polyA signal is preferred.

Host Cells

Any suitable host cell can be used to produce the vectors of the invention. In general, such cells will be transfected mammalian cells but other cell types, eg insect cells, can also be used. In terms of mammalian cell production systems, HEK293 and HEK293T are preferred for AAV vectors. CHO cells may also be used.

Pharmaceutical Compositions and Dosages

Vectors of the invention will typically be presented in the form of a pharmaceutical composition comprising the vector and a pharmaceutically acceptable carrier or excipient. Any suitable carrier or excipient can be used. Dosages and dosage regimes can be determined within the normal skill of the medical practitioner responsible for administration of the composition.

Conditions and Treatments

Sequences and vectors of the invention can be used to treat retinitis pigmentosa, typically by gene therapy techniques. They can be used in combination with other treatments for the same condition or for other conditions

The following Examples illustrate the invention.

### EXAMPLES

### Example 1

### Constructs and Vectors

We have created a modified version of the RPGR-ORF15 gene (RPGR-ORF15*) that has 456 base pairs (152 amino acids) of the highly repetitive sequence removed to improve its stability (FIGS. 1A and 1B).

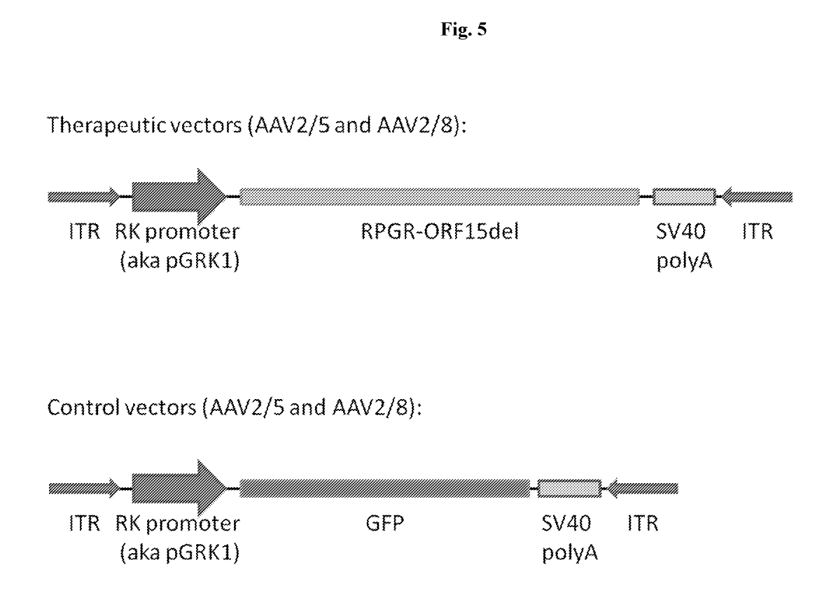

A schematic showing the insertion of the RPGR-ORF15* sequence into therapeutic vectors (AAV2/5 and AAV2/8) is provided in FIG. 5, together with a schematic showing the construction of a control vector containing the reporter gene GFP in place of RPGR-ORF5*.

### Example 2

### Delivery to Mouse Retinas

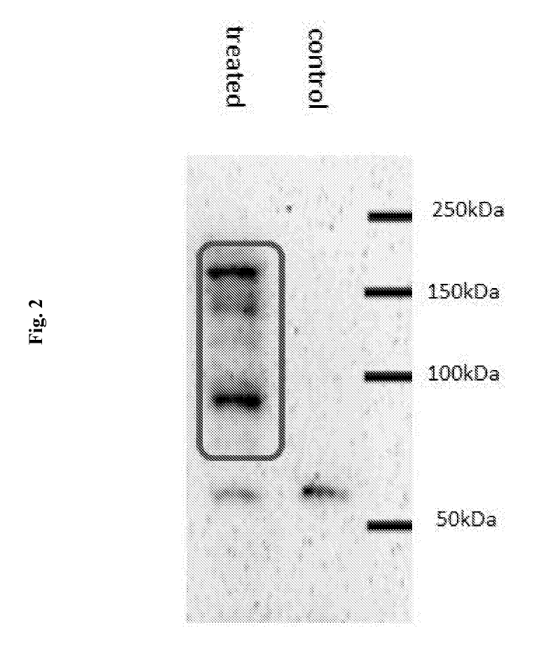

A modified RPGR-ORF15* transgene driven by the human rhodopsin kinase (GRK1) promoter was delivered to retinas using either AAV2/5 (AAV2 genome pseudotyped with an AAVS capsid) or AAV2/8 (id with AAV8 capsid) gene therapy vectors, and proteins were extracted 2 weeks post-treatment. Analysis by protein (western) blot shows the production of RPGR protein of the expected size (FIG. 2), indicating that the new transgene is indeed stable in the context of AAV-based gene therapy vectors.

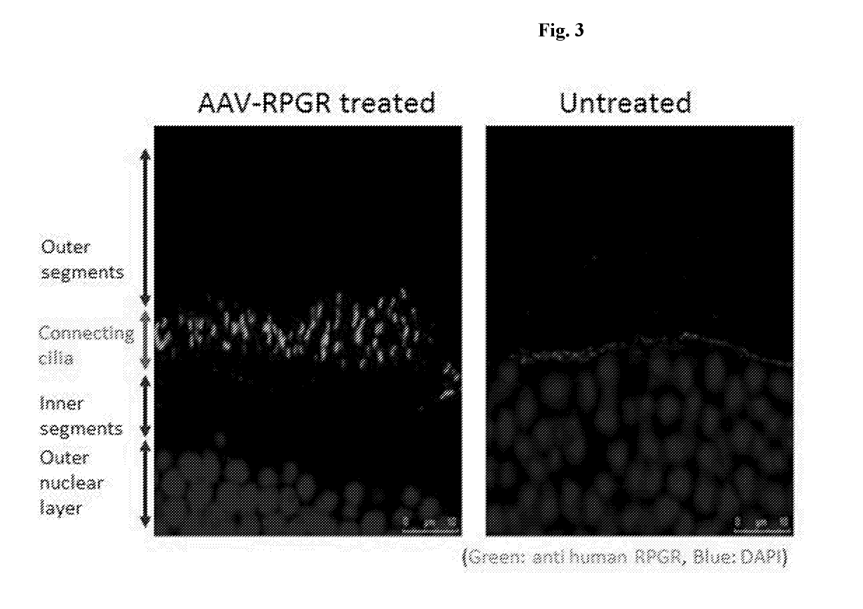

Analysis of the treated RPGR-deficient mouse retinas by immunohistochemistry using an anti-RPGR monoclonal antibody shows that the modified RPGR protein localises correctly to the connecting cilium of the photoreceptor cells (FIG. 3).

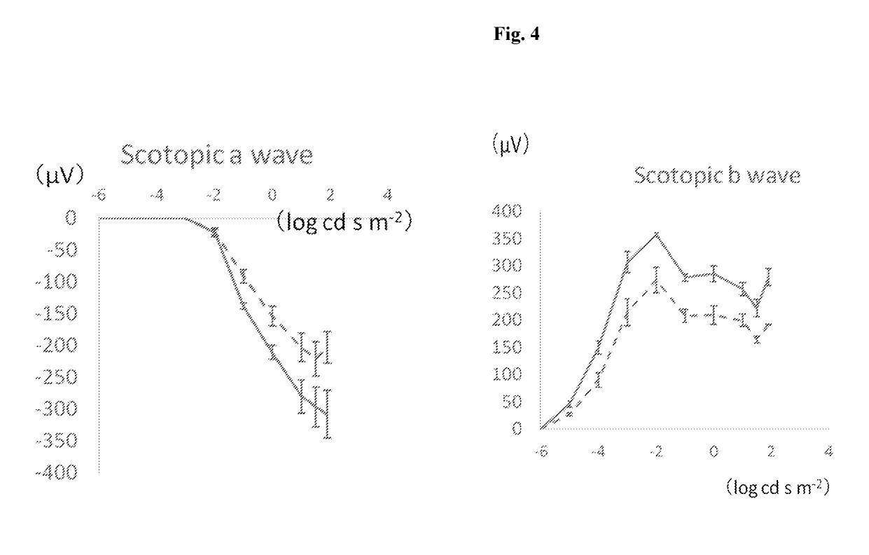

In order to determine whether the modified RPGR-ORF15 transgene product is functional, RPGR-deficient mice were injected with the therapeutic AAV2/8.RPGR-ORF15* construct in the right eyes and an AAV2/8.GFP control vector into the left eyes. Electroretinography analysis of retinal responses to light indicates that the rod photoreceptor cells in the eyes that were treated with this new therapeutic gene therapy vector are more sensitive to light than photoreceptors from eyes treated with a control vector (FIG. 4).

1. A polynucleotide comprising:
(a) the nucleotide sequence shown in SEQ ID NO: 5;
(b) a nucleotide sequence comprising the sequence of SEQ ID NO: 1 but with a deletion corresponding to (i) the sequence of SEQ ID NO:3, (ii) the sequence of SEQ ID NO: 3 and up to 75 additional nucleotides flanking SEQ ID NO:3 on one or both sides of SEQ ID NO:3 in the sequence of SEQ ID NO: 1, or (iii) 390 or more contiguous nucleotides from within SEQ ID NO:3;
(c) a nucleotide sequence according to (a) or (b) but truncated at one or both of its 5′ and 3′ ends by up to 150 nucleotides per end; or
(d) a degenerate nucleotide sequence that encodes the polypeptide sequence of SEQ ID NO: 6 or the same polypeptide as the polynucleotide sequence of any of (a), (b) or (c);
said polynucleotide having the ability to rescue loss of RPGR (retinitis pigmentosa GTPase regulator) function.

2. The polynucleotide according to claim 1, part (b), which is a nucleotide sequence comprising the sequence of SEQ ID NO: 1 but with a deletion corresponding to at least 400, at least 420 or at least 450 contiguous nucleotides of SEQ ID NO:3.

3. A polypeptide comprising an amino acid sequence encoded by a polynucleotide sequence of claim 1 or 2.

4. A vector comprising a polynucleotide of claim 1 or 2 operably linked to a promoter.

5. A vector according to claim 4 which is a viral vector.

6. A viral vector according to claim 5 which is an adeno-associated virus (AAV) vector.

7. A vector of claim 6 which is an AAV2/5 or AAV2/8 vector.

8. A vector according to any one of claims 4 to 7 wherein the promoter is a photoreceptor cell specific promoter.

9. A vector according to claim 8 wherein the promoter is the human rhodopsin kinase (GRK1) promoter.

10. A vector according to any one of claims 4 to 9, further comprising a polyadenylation signal downstream of the sequence of claim 1 or 2.

11. A vector according to claim 10 wherein the polyadenylation signal is an SV40 polyadenylation signal.

12. A host cell that produces a vector of any one of claims 4 to 11.

13. A cell according to claim 12 that is a HEK293 or HEK293T cell

14. A pharmaceutical composition comprising a vector of any one of claims 4 to 11 and a pharmaceutically acceptable carrier.

15. A polynucleotide of claim 1 or 2 or a vector of any one of claims 4 to 11, for use in the treatment of a disease or disorder of the human or animal body.

16. A polynucleotide of claim 1 or 2 or a vector of any one of claims 4 to 11 for use in the treatment of retinitis pigmentosa.

17. Use of a polynucleotide of claim 1 or 2 or a vector of any one of claims 4 to 11 in the manufacture of a medicament for the treatment of retinitis pigmentosa.

18. A method of treating retinitis pigmentosa by administering to a subject in need thereof an effective amount of a polynucleotide of claim 1 or 2, or a vector of any one of claims 4 to 11.


SEQUENCE LISTING (6 sequences)

SEQ ID NO: 1 - DNA, 3459 bp, Artificial Sequence
  SID1
atgagggagccggaagagctgatgcccgattcgggtgctgtgtttacatttgggaaaagtaaatttgctgaaaataatcccggtaaattctggtttaaaaatgatgtccctgtacatctttcatgtggagatgaacattctgctgttgttaccggaaataataaactttacatgtttggcagtaacaactggggtcagttaggattaggatcaaagtcagccatcagcaagccaacatgtgtcaaagctctaaaacctgaaaaagtgaaattagctgcctgtggaaggaaccacaccctggtgtcaacagaaggaggcaatgtatatgcaactggtggaaataatgaaggacagttggggcttggtgacaccgaagaaagaaacacttttcatgtaattagcttttttacatccgagcataagattaagcagctgtctgctggatctaatacttcagctgccctaactgaggatggaagactttttatgtggggtgacaattccgaagggcaaattggtttaaaaaatgtaagtaatgtctgtgtccctcagcaagtgaccattgggaaacctgtctcctggatctcttgtggatattaccattcagcttttgtaacaacagatggtgagctatatgtgtttggagaacctgagaatgggaagttaggtcttcccaatcagctcctgggcaatcacagaacaccccagctggtgtctgaaattccggagaaggtgatccaagtagcctgtggtggagagcatactgtggttctcacggagaatgctgtgtatacctttgggctgggacaatttggtcagctgggtcttggcacttttctttttgaaacttcagaacccaaagtcattgagaatattagggatcaaacaataagttatatttcttgtggagaaaatcacacagctttgataacagatatcggccttatgtatacttttggagatggtcgccacggaaaattaggacttggactggagaattttaccaatcacttcattcctactttgtgctctaattttttgaggtttatagttaaattggttgcttgtggtggatgtcacatggtagtttttgctgctcctcatcgtggtgtggcaaaagaaattgaattcgatgaaataaatgatacttgcttatctgtggcgacttttctgccgtatagcagtttaacctcaggaaatgtactgcagaggactctatcagcacgtatgcggcgaagagagagggagaggtctccagattctttttcaatgaggagaacactacctccaatagaagggactcttggcctttctgcttgttttctccccaattcagtctttccacgatgttctgagagaaacctccaagagagtgtcttatctgaacaggacctcatgcagccagaggaaccagattatttgctagatgaaatgaccaaagaagcagagatagataattcttcaactgtagaaagccttggagaaactactgatatcttaaacatgacacacatcatgagcctgaattccaatgaaaagtcattaaaattatcaccagttcagaaacaaaagaaacaacaaacaattggggaactgacgcaggatacagctcttactgaaaacgatgatagtgatgaatatgaagaaatgtcagaaatgaaagaagggaaagcatgtaaacaacatgtgtcacaagggattttcatgacgcagccagctacgactatcgaagcattttcagatgaggaagtagagatcccagaggagaaggaaggagcagaggattcaaaaggaaatggaatagaggagcaagaggtagaagcaaatgaggaaaatgtgaaggtgcatggaggaagaaaggagaaaacagagatcctatcagatgaccttacagacaaagcagaggtgagtgaaggcaaggcaaaatcagtgggagaagcagaggatgggcctgaaggtagaggggatggaacctgtgaggaaggtagttcaggagcagaacactggcaagatgaggagagggagaagggggagaaagacaagggtagaggagaaatggagaggccaggagagggagagaaggaactagcagagaaggaagaatggaagaagagggatggggaagagcaggagcaaaaggagagggagcagggccatcagaaggaaagaaaccaagagatggaggagggaggggaggaggagcatggagaaggagaagaagaggagggagacagagaagaggaagaagagaaggagggagaagggaaagaggaaggagaaggggaagaagtggagggagaacgtgaaaaggaggaaggagagaggaaaaaggaggaaagagcggggaaggaggagaaaggagaggaagaaggagaccaaggagagggggaagaggaggaaacagaggggagaggggaggaaaaagaggagggaggggaagtagagggaggggaagtagaggaggggaaaggagagagggaagaggaagaggaggagggtgagggggaagaggaggaaggggagggggaagaggaggaaggggagggggaagaggaggaaggagaagggaaaggggaggaagaaggggaagaaggagaaggggaggaagaaggggaggaaggagaaggggagggggaagaggaggaaggagaaggggagggagaagaggaaggagaaggggagggagaagaggaggaaggagaaggggagggagaagaggaaggagaaggggagggagaagaggaggaaggagaagggaaaggggaggaggaaggagaggaaggagaaggggagggggaagaggaggaaggagaaggggaaggggaggatggagaaggggagggggaagaggaggaaggagaatgggagggggaagaggaggaaggagaaggggagggggaagaggaaggagaaggggaaggggaggaaggagaaggggagggggaagaggaggaaggagaaggggagggggaagaggaggaaggggaagaagaaggggaggaagaaggagagggagaggaagaaggggagggagaaggggaggaagaagaggaaggggaagtggaaggggaggtggaaggggaggaaggagagggggaaggagaggaagaggaaggagaggaggaaggagaagaaagggaaaaggagggggaaggagaagaaaacaggaggaacagagaagaggaggaggaagaagaggggaagtatcaggagacaggcgaagaagagaatgaaaggcaggatggagaggagtacaaaaaagtgagcaaaataaaaggatctgtgaaatatggcaaacataaaacatatcaaaaaaagtcagttactaacacacagggaaatgggaaagagcagaggtccaaaatgccagtccagtcaaaacgacttttaaaaaatgggccatcaggttccaaaaagttctggaataatatattaccacattacttggaattgaagtaa

SEQ ID NO: 2 - protein, 1152 aa, Artificial Sequence
  Synthetic Construct
MREPEELMPDSGAVFTFGKSKFAENNPGKFWFKNDVPVHLSCGDEHSAVVTGNNKLYMFGSNNWGQLGLGSKSAISKPTCVKALKPEKVKLAACGRNHTLVSTEGGNVYATGGNNEGQLGLGDTEERNTFHVISFFTSEHKIKQLSAGSNTSAALTEDGRLFMWGDNSEGQIGLKNVSNVCVPQQVTIGKPVSWISCGYYHSAFVTTDGELYVFGEPENGKLGLPNQLLGNHRTPQLVSEIPEKVIQVACGGEHTVVLTENAVYTFGLGQFGQLGLGTFLFETSEPKVIENIRDQTISYISCGENHTALITDIGLMYTFGDGRHGKLGLGLENFTNHFIPTLCSNFLRFIVKLVACGGCHMVVFAAPHRGVAKEIEFDEINDTCLSVATFLPYSSLTSGNVLQRTLSARMRRRERERSPDSFSMRRTLPPIEGTLGLSACFLPNSVFPRCSERNLQESVLSEQDLMQPEEPDYLLDEMTKEAEIDNSSTVESLGETTDILNMTHIMSLNSNEKSLKLSPVQKQKKQQTIGELTQDTALTENDDSDEYEEMSEMKEGKACKQHVSQGIFMTQPATTIEAFSDEEVEIPEEKEGAEDSKGNGIEEQEVEANEENVKVHGGRKEKTEILSDDLTDKAEVSEGKAKSVGEAEDGPEGRGDGTCEEGSSGAEHWQDEEREKGEKDKGRGEMERPGEGEKELAEKEEWKKRDGEEQEQKEREQGHQKERNQEMEEGGEEEHGEGEEEEGDREEEEEKEGEGKEEGEGEEVEGEREKEEGERKKEERAGKEEKGEEEGDQGEGEEEETEGRGEEKEEGGEVEGGEVEEGKGEREEEEEEGEGEEEEGEGEEEEGEGEEEEGEGKGEEEGEEGEGEEEGEEGEGEGEEEEGEGEGEEEGEGEGEEEEGEGEGEEEGEGEGEEEEGEGKGEEEGEEGEGEGEEEEGEGEGEDGEGEGEEEEGEWEGEEEEGEGEGEEEGEGEGEEGEGEGEEEEGEGEGEEEEGEEEGEEEGEGEEEGEGEGEEEEEGEVEGEVEGEEGEGEGEEEEGEEEGEEREKEGEGEENRRNREEEEEEEGKYQETGEEENERQDGEEYKKVSKIKGSVKYGKHKTYQKKSVTNTQGNGKEQRSKMPVQSKRLLKNGPSGSKKFWNNILPHYLELK

SEQ ID NO: 3 - DNA, 456 bp, Artificial Sequence
  SID2
gaagaggaggagggtgagggggaagaggaggaaggggagggggaagaggaggaaggggagggggaagaggaggaaggagaagggaaaggggaggaagaaggggaagaaggagaaggggaggaagaaggggaggaaggagaaggggagggggaagaggaggaaggagaaggggagggagaagaggaaggagaaggggagggagaagaggaggaaggagaaggggagggagaagaggaaggagaaggggagggagaagaggaggaaggagaagggaaaggggaggaggaaggagaggaaggagaaggggagggggaagaggaggaaggagaaggggaaggggaggatggagaaggggagggggaagaggaggaaggagaatgggagggggaagaggaggaaggagaaggggagggggaagaggaaggagaaggggaaggggaggaaggagaaggggag

SEQ ID NO: 4 - protein, 152 aa, Artificial Sequence
  Synthetic Construct
EEEEGEGEEEEGEGEEEEGEGEEEEGEGKGEEEGEEGEGEEEGEEGEGEGEEEEGEGEGEEEGEGEGEEEEGEGEGEEEGEGEGEEEEGEGKGEEEGEEGEGEGEEEEGEGEGEDGEGEGEEEEGEWEGEEEEGEGEGEEEGEGEGEEGEGE

SEQ ID NO: 5 - DNA, 3003 bp, Artificial Sequence
  SID3
atgagggagccggaagagctgatgcccgattcgggtgctgtgtttacatttgggaaaagtaaatttgctgaaaataatcccggtaaattctggtttaaaaatgatgtccctgtacatctttcatgtggagatgaacattctgctgttgttaccggaaataataaactttacatgtttggcagtaacaactggggtcagttaggattaggatcaaagtcagccatcagcaagccaacatgtgtcaaagctctaaaacctgaaaaagtgaaattagctgcctgtggaaggaaccacaccctggtgtcaacagaaggaggcaatgtatatgcaactggtggaaataatgaaggacagttggggcttggtgacaccgaagaaagaaacacttttcatgtaattagcttttttacatccgagcataagattaagcagctgtctgctggatctaatacttcagctgccctaactgaggatggaagactttttatgtggggtgacaattccgaagggcaaattggtttaaaaaatgtaagtaatgtctgtgtccctcagcaagtgaccattgggaaacctgtctcctggatctcttgtggatattaccattcagcttttgtaacaacagatggtgagctatatgtgtttggagaacctgagaatgggaagttaggtcttcccaatcagctcctgggcaatcacagaacaccccagctggtgtctgaaattccggagaaggtgatccaagtagcctgtggtggagagcatactgtggttctcacggagaatgctgtgtatacctttgggctgggacaatttggtcagctgggtcttggcacttttctttttgaaacttcagaacccaaagtcattgagaatattagggatcaaacaataagttatatttcttgtggagaaaatcacacagctttgataacagatatcggccttatgtatacttttggagatggtcgccacggaaaattaggacttggactggagaattttaccaatcacttcattcctactttgtgctctaattttttgaggtttatagttaaattggttgcttgtggtggatgtcacatggtagtttttgctgctcctcatcgtggtgtggcaaaagaaattgaattcgatgaaataaatgatacttgcttatctgtggcgacttttctgccgtatagcagtttaacctcaggaaatgtactgcagaggactctatcagcacgtatgcggcgaagagagagggagaggtctccagattctttttcaatgaggagaacactacctccaatagaagggactcttggcctttctgcttgttttctccccaattcagtctttccacgatgttctgagagaaacctccaagagagtgtcttatctgaacaggacctcatgcagccagaggaaccagattatttgctagatgaaatgaccaaagaagcagagatagataattcttcaactgtagaaagccttggagaaactactgatatcttaaacatgacacacatcatgagcctgaattccaatgaaaagtcattaaaattatcaccagttcagaaacaaaagaaacaacaaacaattggggaactgacgcaggatacagctcttactgaaaacgatgatagtgatgaatatgaagaaatgtcagaaatgaaagaagggaaagcatgtaaacaacatgtgtcacaagggattttcatgacgcagccagctacgactatcgaagcattttcagatgaggaagtagagatcccagaggagaaggaaggagcagaggattcaaaaggaaatggaatagaggagcaagaggtagaagcaaatgaggaaaatgtgaaggtgcatggaggaagaaaggagaaaacagagatcctatcagatgaccttacagacaaagcagaggtgagtgaaggcaaggcaaaatcagtgggagaagcagaggatgggcctgaaggtagaggggatggaacctgtgaggaaggtagttcaggagcagaacactggcaagatgaggagagggagaagggggagaaagacaagggtagaggagaaatggagaggccaggagagggagagaaggaactagcagagaaggaagaatggaagaagagggatggggaagagcaggagcaaaaggagagggagcagggccatcagaaggaaagaaaccaagagatggaggagggaggggaggaggagcatggagaaggagaagaagaggagggagacagagaagaggaagaagagaaggagggagaagggaaagaggaaggagaaggggaagaagtggagggagaacgtgaaaaggaggaaggagagaggaaaaaggaggaaagagcggggaaggaggagaaaggagaggaagaaggagaccaaggagagggggaagaggaggaaacagaggggagaggggaggaaaaagaggagggaggggaagtagagggaggggaagtagaggaggggaaaggagagagggaagagggggaagaggaggaaggagaaggggagggggaagaggaggaaggggaagaagaaggggaggaagaaggagagggagaggaagaaggggagggagaaggggaggaagaagaggaaggggaagtggaaggggaggtggaaggggaggaaggagagggggaaggagaggaagaggaaggagaggaggaaggagaagaaagggaaaaggagggggaaggagaagaaaacaggaggaacagagaagaggaggaggaagaagaggggaagtatcaggagacaggcgaagaagagaatgaaaggcaggatggagaggagtacaaaaaagtgagcaaaataaaaggatctgtgaaatatggcaaacataaaacatatcaaaaaaagtcagttactaacacacagggaaatgggaaagagcagaggtccaaaatgccagtccagtcaaaacgacttttaaaaaatgggccatcaggttccaaaaagttctggaataatatattaccacattacttggaattgaagtaa

SEQ ID NO: 6 - protein, 1000 aa, Artificial Sequence
  Synthetic Construct
MREPEELMPDSGAVFTFGKSKFAENNPGKFWFKNDVPVHLSCGDEHSAVVTGNNKLYMFGSNNWGQLGLGSKSAISKPTCVKALKPEKVKLAACGRNHTLVSTEGGNVYATGGNNEGQLGLGDTEERNTFHVISFFTSEHKIKQLSAGSNTSAALTEDGRLFMWGDNSEGQIGLKNVSNVCVPQQVTIGKPVSWISCGYYHSAFVTTDGELYVFGEPENGKLGLPNQLLGNHRTPQLVSEIPEKVIQVACGGEHTVVLTENAVYTFGLGQFGQLGLGTFLFETSEPKVIENIRDQTISYISCGENHTALITDIGLMYTFGDGRHGKLGLGLENFTNHFIPTLCSNFLRFIVKLVACGGCHMVVFAAPHRGVAKEIEFDEINDTCLSVATFLPYSSLTSGNVLQRTLSARMRRRERERSPDSFSMRRTLPPIEGTLGLSACFLPNSVFPRCSERNLQESVLSEQDLMQPEEPDYLLDEMTKEAEIDNSSTVESLGETTDILNMTHIMSLNSNEKSLKLSPVQKQKKQQTIGELTQDTALTENDDSDEYEEMSEMKEGKACKQHVSQGIFMTQPATTIEAFSDEEVEIPEEKEGAEDSKGNGIEEQEVEANEENVKVHGGRKEKTEILSDDLTDKAEVSEGKAKSVGEAEDGPEGRGDGTCEEGSSGAEHWQDEEREKGEKDKGRGEMERPGEGEKELAEKEEWKKRDGEEQEQKEREQGHQKERNQEMEEGGEEEHGEGEEEEGDREEEEEKEGEGKEEGEGEEVEGEREKEEGERKKEERAGKEEKGEEEGDQGEGEEEETEGRGEEKEEGGEVEGGEVEEGKGEREEGEEEEGEGEGEEEEGEEEGEEEGEGEEEGEGEGEEEEEGEVEGEVEGEEGEGEGEEEEGEEEGEEREKEGEGEENRRNREEEEEEEGKYQETGEEENERQDGEEYKKVSKIKGSVKYGKHKTYQKKSVTNTQGNGKEQRSKMPVQSKRLLKNGPSGSKKFWNNILPHYLELK